(19) United States
(12) Patent Application Publication
(10) Pub. No.: US 2005/0113720 A1
(43) Pub. Date: May 26, 2005

(54) Method and device for determining the center of a joint

(76) Inventors: Philippe Cinquin, La Tronche (FR); Laurent Desbat, Grenoble (FR); Stephane Lavallee, La Tronche (FR)

Correspondence Address: DUANE MORRIS LLP, PO BOX 5203, PRINCETON, NJ, 08543-5203, US

(21) Appl. No.: 11/032,740

(22) Filed: Jan. 11, 2005

(30) Foreign Application Priority Data
Nov. 10, 1998 (FR) 98/14298
Nov. 8, 1999 (WO) PCT/FR99/02733

(51) Int. Cl. A61B 5/103; A61B 5/117; A61B 19/00

(52) U.S. Cl. 600/587; 128/898

(57) ABSTRACT

A method for determining the center of rotation of a bone in a revolute joint, for example, a femur in the iliac bone, including the steps of displacing said bone, locating several ones of its positions, and memorizing them, imposing a constraint to the displacement of said center of rotation without for all this immobilizing it, and searching a point linked to the referential of said bone for which an optimization criterion taking into account said constraint is reached.

### CROSS REFERENCE TO RELATED APPLICATIONS

This application is a continuation-in-part of U.S. patent application Ser. No. 09/831,170, filed Aug. 20, 2001.

### BACKGROUND

The present invention aims at locating the center of rotation of a rigid organ with respect to a determined point of this organ. The present invention finds applications in complex mechanical systems where it is practically impossible to determine by direct calculation the motion of given organs with respect to other. It especially finds applications in the case of rigid organs of the human body, such as bones, and will be more specifically described hereafter in the context of the determination of the center of rotation of a revolute joint, and more specifically still in the context of the determination of the center of a femoral head.

For many human body motion analysis, diagnosis, or surgical operations, it is previously required to accurately determine the position of the center of a femoral head with respect to a referential linked to a patient's femur or pelvis. It should be noted that this determination in itself is not a diagnosis operation, nor a medical or surgical operation. It has no effect on the considered organ and may be performed on a healthy organ, to analyze its motion and, for example, foresee the athletic capacities of an individual. Further, even if it is used with a view to diagnosis or with a medical or surgical aim, it is only an accessory thereof, in the same way as a doctor needs to know the size and weight of a patient as diagnosis elements.

A method for determining the center of rotation of a femur with respect to the pelvis is for example described in international patent application WO-98/40037 published on Sep. 17, 1998, and assigned to Aesculap Company.

### DETAILED DESCRIPTION OF THE DRAWING

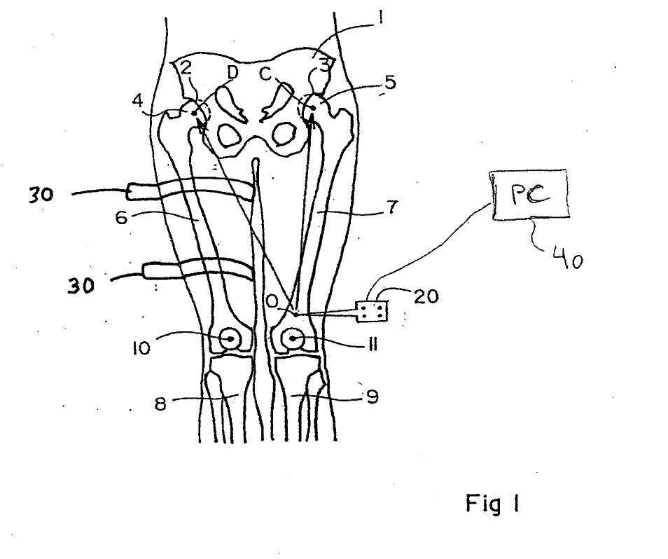

FIG. 1 is a schematic illustration of an embodiment of the device of the present invention shown in conjunction with a patient.

### DESCRIPTION

This patent application provides, to determine the center of rotation of a femur with respect to a pelvis, use of several markers provided with light-emitting diodes driven into bones of the patient. The markers are associated with known systems of localization by triangulation. A first marker is fastened in the femur and a second marker is fastened in the iliac bone. The femur is moved according to several positions. Each of the positions of the first marker is detected by using a triangulation system and is stored in a calculator, taking into account the displacement of the marker fastened in the iliac bone. The invariant distance between the marker linked to the femur and the center of rotation of the femoral head can then be searched by various conventional mathematical minimization methods using for example least error squares algorithms.

This system has provided full satisfaction, but it has the disadvantage of requiring implantation of rigid objects into the femur and the iliac bone and thus provision of incisions.

The present invention aims at avoiding at least the implantation in the iliac bone. To achieve this object, the present invention provides a method for determining the center of rotation of a bone in a revolute joint, including the steps of displacing said bone, locating several ones of its positions, and memorizing them; imposing a constraint to the displacement of said center of rotation without for all this immobilizing it; and searching a point linked to the referential of said bone for which an optimization criterion taking into account said constraint is reached.

According to an embodiment of the present invention, applied to the determination of the center of rotation of a first femur with respect to the iliac bone, the method includes the steps of immobilizing the second femur, displacing the first femur and locating several ones of its positions, and searching the invariants of this displacement, taking into account the fact that the centers of rotations of the first and second femurs are distant by a substantially constant length.

According to an embodiment of the present invention, the method further includes the step of locating upon each measurement of the position of the first femur the position of the second femur to accordingly correct the position of the center of rotation between the first femur and the iliac bone.

According to an embodiment of the present invention, applied to determining the center of rotation of a first femur with respect to the iliac bone, the method includes the steps of displacing the thigh so that said center of rotation moves along a trajectory which is clearly mathematically distinct from all other points of the lower femur portion, and searching this point having a specific trajectory by an optimization method.

According to an embodiment of the present invention, the thigh is moved so that the knee follows a loop trajectory, whereby only the trajectory of the center of rotation will optimize a distance in the expression of which the number of loops and some of their mathematical characteristics will be involved.

According to an embodiment of the present invention, the thigh motion can be decomposed in several elementary motions, for each elementary motion, an optimal center of rotation and an optimized distance value are calculated, and the center of rotation is statistically defined, taking into account each of the estimations of the center of rotation and of the optimized distance value, obtained based on each of the elementary motions.

According to an embodiment of the present invention, applied to determining the center of rotation of a first femur with respect to the iliac bone, the method includes the steps of moving the thigh so that its lower portion describes as simple a trajectory as possible, including, in particular, no loops, so that the searched center of rotation describes a mathematically simple trajectory, and searching this point with a mathematically simple trajectory by an optimization method.

According to an embodiment of the present invention, the thigh motion can be decomposed in several elementary motions, for each elementary motion, an optimal center of rotation and the value of the optimized distance are calculated, and the center of rotation is statistically defined, by taking into account each of the estimations of the center of rotation and of the value of the optimized distance, obtained based on each of the elementary motions.

According to an embodiment of the present invention, applied to determining the center of rotation of a first femur with respect to the iliac bone, the method includes the steps of performing a succession of elementary motions of the thigh, for each of these motions, searching the position of the center of rotation of the femur, assuming that said femur has remained fixed, and determining a confidence ellipsoid within which the probability of presence of the femur center of rotation is high, and calculating based on the confidence ellipsoids the position of maximum probability of the femur center of rotation.

According to an embodiment of the present invention, some of the elementary motions of the thigh are performed in a plane and are of small amplitude. According to an embodiment of the present invention, some of the elementary motions of the thigh are performed by rotating the femur around its own axis.

The present invention also provides a device for determining the center of rotation of a femur with respect to the iliac bone, including means for locating several positions of the femur during motions thereof, means for imposing a constraint to the motion of said center of rotation without for all this immobilizing it, and calculation means for searching a point linked to the referential of said femur for which a minimization criterion is reached, taking said constraint into account.

The foregoing objects, features and advantages of the present invention will be discussed in detail in the following non-limiting description of specific embodiments in connection with the accompanying drawing, which very schematically shows a lower portion of the person's skeleton.

More specifically, the single drawing shows iliac bone 1 and its right 2 and left 3 acetabuli (it should be noted that the right acetabulum appears on the left-hand side of the drawing, which is a front view) in which are engaged heads 4 and 5 of right and left femurs 6 and 7. The starting of tibias 8 and 9 and kneecaps 10 and 11 have also been shown.

A simple way of determining the center of rotation of a femur, that is, substantially the center of femoral head, would consist, as indicated in the above-mentioned patent application, of measuring several successive positions of the femur while the pelvis, and more specifically the iliac bone, are immobilized. A vector having an invariant top can thus be determined and the end of this vector indicates the center of rotation.

The locating of the femur position may be performed in various ways. In particular, systems for locating the position of transmitters—such as optical or infrared transmitters, but which could also be transmitters radiating at other wavelength or magnetic transmitters—which use sets of sensors and determine the position of each of the transmitters by triangulation, are known. An example of such an installation applied to the determination of the head position is described in the article of Innovation et Technologie en Biologie et Médecine (ITBM) journal, volume 13, No 4, 1992, by L. Adams et al., pages 410-424. There also exist systems sold under trade name “Optotrak” by Northern Digital Company.

Unfortunately, such a simple system poorly operates since it is very difficult to immobilize the pelvis of a patient laying on his back and, when his leg is moved, especially due to the elasticity of the skin and muscles between the iliac bone and the table on which the patient lies, the pelvis is inevitably moved. Thus, as described in the preceding patent application, a second localization system or marker inserted in the iliac bone has to be used. This requires performing an incision into the skin and piercing the iliac bone to fixedly position a marker therein. The present invention essentially aims at suppressing this step.

The present invention provides a system that avoids implantation of a marker into the iliac bone and which does not require perfect pelvis immobilization. Generally, the present invention provides imposing constraints to the pelvis motions by various physical processes and deducing from these physical constraints mathematical characteristics of the trajectory of the center of rotation enabling identification thereof.

The invention uses a computer to store the measurements, perform the computations, etc.

In all the described embodiments, the location of the femur position can be performed in various ways using known systems for locating the position of transmitters. For example, a system using two charge-coupled-detector (CCD) cameras to triangulate the position of active markers as disclosed in the article of Adams et al. in ITBM journal above mentioned can be used. In such a system, a series of small infrared lights emit pulses sequentially, and the cameras are synchronized with the emitters so that each of the cameras receives only one specific signal at a time. The location of each light on each 2D camera image defines a 3D line in space. Using at least two cameras permits the acquisition of 3D coordinates of an emitter (marker) attached to the femur by computing the intersection of the two corresponding projection lines.

Another example is to use retro-reflective passive markers as disclosed in the article “Multimodal Information for CIS” of S. Lavallée et al. published in Computer Integrated Surgery, MIT Press in 1996. In such a passive system, each marker simultaneously reflects infrared light back to the camera sensor. The segmentation of reference features (passive targets) in images and the computation of 3D coordinates from this image points is made by a conventional computer vision system. As for the previous example, a first marker is attached to the femur and a second marker is attached to an object of the room, for example, the bed on which lays the patient.

Of course, other known localization systems can be used provided that a marker (active or passive) of the system can be attached to the femur. Usually, another marker is to be attached to an object in the room for referential conversion purposes.

In a first embodiment the present invention is based on the two following observations. The first observation is that, if it is very difficult to immobilize the pelvis of a patient lying on his back, it is however possible to immobilize his thigh, and thus his femur, by mechanical system 30, pneumatic or vacuum systems, which compress and block the thigh or knee. The second observation is that, given the structure of the human body, it is possible to fasten an external marker against the femoral condyles, the position of this marker remaining perfectly fixed with respect to the femur.

In this first embodiment of the present invention, to measure the position of the center of a femur head, for example left femur head 5, the patient's opposite thigh, the one containing femur 6, is secured or immobilized. With the patient laying on a bed, his thigh opposite to which containing the femur 7, whose femur head 5 is to be located, is secured or immobilized, for example with belts or straps 30, without securing the thigh containing the femur 7.

Thus, right femur head 4 remains in a fixed position. As the iliac bone is linked to femur 6 of the immobilized thigh, the constraints are transmitted to the other leg through the iliac bone and the femur head 5. The only possible motions of the iliac bone then are rotation motions around this femur head. Hence, the displacements of the center of rotation (center of the femur head 5) of the femur 7 with respect to the iliac bone are limited even if this center of rotation is not immobile. Designating by C the center of rotation of head 5 of femur 7 and by D the center of rotation of head 4 of femur 6, since point D is fixed, point C can only move on a sphere centered on point D. Thus, a point O attached to femur 7 can only move according to a combination of motions including a rotation of fixed radius around point C, and a rotation of fixed radius of point C around point D.

Displacement of the femur 7 in order to obtain several measurements of the position of the center of rotation C can be achieved in different ways. For example, an operator takes the left leg of the patient and manually imposes displacements of this leg.

Knowing several positions of point O and the corresponding femur orientations, as determined by means 20 for locating several positions of the femur during motion thereof, the problem to be solved to determine the position of point C is an optimization problem. For example, with a marker attached to the pelvis and to the distal femur, when moving the femur relative to the pelvis, a marker on the femur generates points on a virtual sphere when represented in the pelvic coordinate frame. The center of this sphere is calculated by a least-squares algorithm. Such an exemplary method is disclosed in the article “Computer-Assisted Surgical Total Knee Replacement” of Leitner et al. published by Springer in the proceedings of the first Joint Conference of CVRMed. And MRCAS, Grenoble, France 1997.

Various calculation methods implemented in a computer 40 (which can be shared with the localization device) for solving this problem may be used: general non-linear least error squares methods, methods adapted to cases where the expression to be minimized is a square of sums of squares, formal calculation methods for solving polynomial equation systems . . .

Descriptions of these and other methods can be found in the following works:

NAG program library, Numerical Algorithms Group ldt, Wilkinson House, Jordan Hill road, Oxford, UK OX2 8DR,

IMSL program library, International Mathematical and Statistical Library, Visual Numerics inc., 9990 Richmond Suite 4000, Houston, Tex. 77042, USA,

Régression non linéaire et applications, A. Antoniadis et al., “Economie et Statistiques Avancées” collection, Economica, 1992, France,

Introduction à l'analyse numérique matricielle et à l'optimisation, Masson, 1982, France.

Mathematical methods enabling determination of the positions of centers of rotations C and D also enable determination of a certain uncertainty on the result. Especially, a residue indicating whether the considered points really are fixed points appears. If this residue appears to be too large, this means that femur 6 has not been properly immobilized and that point D has moved in the patient's manipulation. To overcome this disadvantage, a marker may be attached to femur 6. As indicated previously, such a marker needs not penetrate into the bone, but can be attached outside of the leg, for example close to the knee against the femoral condyles close to kneecap 10. Thus, for each position of femur 7, the displacement of femur 6, and thus of point D, can be determined to perform the corresponding correction.

The present invention also provides other means for determining the position of the center of a femur head without requiring driving of a marker into the iliac bone and without requiring immobilization of the pelvis or of the opposite femur 6, or following its motions. In each of the following embodiments, the position of the femur of which the center of rotation is desired to be located is tracked by a marker and triangulation system of the previously described type.

According to a second embodiment of the present invention, motions of the thigh such that the center of rotation of the femur always moves along a trajectory which is clearly distinct from that of the other points of the lower femur end are performed, and the point having a trajectory that maximizes a distance to the trajectory of the marker attached to the lower part of the femur is searched, by an optimization method of the above-mentioned type. The distance between trajectories may take into account “topological” features (for example, and non-limitingly: number of self-intersections in the trajectory, or in the projections of the trajectory on given sub-spaces such as planes or spheres; number of “loops” thus delimited; relative positions of the covered loops while “looking at” the inside or the outside of said loops, inside and outside being understood in the sense of “Ampere's rule”, which is conventional in electricity; number and relative positions of points having specific topological features, such as for example stationary points; etc.) or “energetic” features (for example, and non-limitingly: trajectory length; flexion energy of the trajectory, a conventional linear approximation of which is the integral of the square of the second derivative—see Approximation et Optimisation, Pierre-Jean Laurent, Herman 1972; and generally an integral of expressions involving the curve derivatives at orders ranging to the 3^{rd} order or more; etc.). It will be attempted to move the patient's knee so that its lower end describes complex trajectories. Indeed, the center of rotation will not be able to “follow” these complex motions, and will thus describe a mathematically simpler trajectory, which will enable identifying it. For example, the patient's knee will be moved so that it follows a trajectory including at least one “crossing”, an eight for example, or a succession of loops. Then, the trajectories of most femur points will have the shape of an eight or will more generally include one or several crossing points. Only the trajectory of points close to the center of rotation, or even, in certain cases, of the sole center of rotation, will have no crossing point, or less than the trajectory of the reference marker. Thus, even if the center of rotation is not fixed, this center will be able to be identified with respect to point O as being the only point having a trajectory optimizing a distance to the trajectory of the reference marker constructed based on the previously-defined “topological” or “energetic” criteria.

Three examples of the above energetic criteria based methods are summarized bellow. In all these examples, the following notations will be used:
n, number of measurements;
k, time index at which the position M(k) of the marker is measured (M representing a triplet of three coordinates in the referential R of the localization device);
A(k), the rotation matrix 3×3 with respect to the referential R;
Rk, the referential of the femur (at time k) having the point M(k) as origin and the three vectors of which correspond to the three column vectors of the matrix A(k).

With these notations, the position of the center of rotation C(k) (triplet of coordinates) in the referential Rk can be expressed:
C(k)=M(k)+A(k)·C.

At time k+1, the position C(k+1) can be written:
C(k+1)=M(k+1)+A(k+1)·C.

According to a first example, the energetic criterion is the length of the trajectory of a point of the femur in the referential R. The operator imposes various displacements to the leg of the patient. The trajectory of any point of the femur can be expressed as the combination of the displacement of the rotation center and the displacement of the femur with respect to its rotation center. As the patient himself does not move (for example, he is laying on a table), the rotation center can be considered as the only point for which the second part (displacement with respect to the rotation center) is null. Hence, searching the position of rotation center corresponds to search the point of the femur with the minimal displacement in the referential R.

The distance F to be optimized (minimized) varies with the length of the trajectory, and vanishes when this length is zero. This distance can be expressed:
F=Σ [symbol] C(k)·C(k+1), C(k)·C(k+1) [symbol] =Σ [symbol] Fk,Fk [symbol] ,
where <a, b> designates the scalar product between the two vectors a and b. Further:
Fk=C(k)·C(k+1)=M(k)·M(k+1)+[A(k+1)−A(k)]·C.

Noting Nk the vector M(k)·M(k+1) and Bk the matrix [A(k+1)−A(k)], Fk=Nk+Bk·C.

The Minimization of the distance F is, for example, obtained by applying a “linear least-square” method to F expressed as [symbol] U+TC, U+TC [symbol] , i.e. ∥U+TC∥², where U designates le 3n dimensional vector formed by all vectors Nk and T the 3n×3 matrix formed by all matrix Bk.

Minimization methods are well known in the art (for example, “Approximation and Optimisation” from Pierre-Jean Laurent above mentioned or “Numerical Recipes in C” from William H. Hess, Brian P. Flannery, Saul A. Teukolsky and William T. Vetterling, Cambridge University Press).

According to a second example, the energetic criterion is the flexion energy. As the trajectory of the center of rotation is much more “erratic” than the trajectory of the others points, minimizing the flexion energy (more exactly, its opposite) will lead to the position of the center of rotation.

The flexion energy E can be expressed:

E=Σ [symbol] C″(k)·C″(k) [symbol] , where C″ (k) designates the second derivative of C with respect to the measurement time k.

Assuming that the measurement have been made at periodic times, the first derivative C′ (k) of C with respect to the time k can be approximated to C(k)−C(k+1). Then, the second derivative C″ (k) can be approximated to C′(k)·C′(k+1). Minimizing the flexion energy corresponds to minimize the sum Σ [symbol] C′(k)−C′(k+1), C′(k)·C′(k+1) [symbol] with a linear least-square method as above.

Alternatively, the time is replaced by the curvilinear abscissa noted s(k). Then the expression to be minimized is:

$$\frac{Σ 〈 {C}^{′} ( k ) \cdot {C}^{′} ( k + 1 ) , {C}^{′} ( k ) \cdot {C}^{′} ( k + 1 ) 〉}{[ s ( k ) \cdot s ( k + 1 ) ]} .$$

This minimization can be made by applying a non linear least square method such as the known “Levenberg-Marquardt” method (“Numerical Recipes in C” above mentioned).

According to a third example, the energetic criterion is the variance of the trajectory. As the displacements of the iliac bone are limited, when the femur is displaced by the operator, its rotation center defines a cluster of points around its gravity center. On the contrary, the other points of the femur follow a more dispersed set of points. Using the variance to characterize the dispersion of the cluster of points, it is possible to apply a minimization method to this variance to obtain the position of the center of rotation.

Noting G the barycentre of a cluster of n measured points, the variance V can be expressed as:

$$V = Σ 〈 G \cdot C ( k ) , G \cdot C ( k ) 〉 , where G = \frac{1}{n} ΣC ( k ) . Further : \\ G \cdot c ( k ) = C ( k ) - \frac{1}{n} {\sum }_{m = 1}^{n} C ( m ) , where 1 \leq m \leq n . Then : \\ G \cdot C ( k ) = M ( k ) - \frac{1}{n} {\sum }_{m = 1}^{n} M ( m ) + [ A ( k ) - \frac{1}{n} {\sum }_{m = 1}^{n} A ( m ) ] \cdot C . \\ Hence : \\ G \cdot C ( k ) = N ( k ) + B ( k ) \cdot C , with N ( k ) = M ( k ) - \frac{1}{n} {\sum }_{m = 1}^{n} M ( m )$$

$$and B ( k ) = A ( k ) - \frac{1}{n} {\sum }_{m = 1}^{n} A ( m ) .$$

The minimization of the variance then involves the same linear least-squares method as above.

According to an embodiment, if the calculations do not lead to an acceptable determination of the center (for example, if the potential error is too high), the operator is informed by the system so that he can do the process again with other displacements of the femur.

According to a third embodiment of the present invention, not one, but several trajectories such as those corresponding to the second mode are performed. The processing of the data characterizing each of these trajectories is performed according to the second mode, which provides several estimates of the center of rotation. The quality of each of these estimates can be estimated by the value of the used optimization criterion. The point retained as a final estimate of the center of rotation is the result of a statistical processing of this set of estimates, taking into account quality indicators of these estimates (for example, weighted average, non-linear statistical processings, median filtering, etc.).

According to a fourth embodiment of the present invention, motions of the thigh such that its lower part moves according to as “simple” a trajectory as possible, that is, exhibiting none of the topological features used in the second embodiment, and in particular no loops, are performed. The center of rotation of the femur will then be determined as being the femur point having a trajectory minimizing the “energetic” criteria introduced in the description of the second mode. The same optimization methods may be applied. This determination of the center of rotation may however depend on the way in which the thigh rotating motions are transmitted to the iliac bone. Now, this transmission depends on the way in which is urged the thigh, on which compression (force pushing the femur to the pelvis) or traction (force tending to draw the femur away from the pelvis) may simultaneously be exerted for a given stress causing the rotation. To eliminate this effect, motions of the thigh alternating compression and traction may thus be performed.

According to a fifth embodiment of the present invention, not one, but several trajectories such as those corresponding to the fourth mode are followed. The processing of the data characterizing each of these trajectories is performed according to the fourth mode, which provides several estimates of the center of rotation. The quality of each of these estimates can be estimated by the value of the used optimization criterion. The point retained as a final estimate of the center of rotation is the result of a statistical processing of this set of estimates, taking into account quality indicators of these estimates (for example, weighted average, non-linear statistical processings, median filtering, etc.).

According to a sixth embodiment of the present invention, a succession of elementary motions of the thigh are performed. For each of these motions, the position of the femur center of rotation is searched, assuming that it has remained fixed, and a confidence ellipsoid within which the probability of presence of the femur center of rotation is high, for example greater than 95%. Based on several of these confidence ellipsoids, the position of maximum probability of the femur center of rotation is calculated. Each of the confidence ellipsoids is estimated in a referential linked to the femur: possible motions of the femur between the elementary motions are thus allowed for and will not adversely affect the accuracy of the determination of the searched center of rotation. As an example of motions of the thigh adapted to implementing this method, motions with a low urge of the ligamentary, capsular, and muscular apparatus ensuring the cohesion between the femur and the pelvis may be chosen. Such motions are for example rotating motions of the femur around its axis, or motions where the femur end moves with a sufficiently limited amplitude, describing for example approximately in a plane a portion of a circle, said plane being likely to contain, for example, approximately the center of rotation or to be approximately perpendicular to the axis formed by the center of rotation and the center of said circle. For this method to be operative, each of the confidence ellipsoids must be sufficiently small, as least in one dimension. Given that present calculators provide such ellipsoids practically in real time, if, after a motion, too large an ellipsoid is obtained, the operator will cancel the obtained result and perform a new motion, for example, of smaller amplitude or according to one of the other suggested modes. Methods based on Confidence ellipsoids are disclosed, for example, “Numerical Recipes in C” above mentioned, chapter 14.

The present invention has been described in detail in relation with a method for determining the center of rotation of a femur. It should be noted that, except for the first described embodiment, it more generally applies to the determination of the center of rotation of a bone in a revolute joint.

1. A method for determining the center of rotation of a first femur in a revolute joint of an iliac bone, the method comprising:
displacing said first femur, locating several ones of said first femur's positions, and memorizing said positions;
imposing a constraint to the displacement of said center of rotation of said first femur without immobilizing said first femur; and
searching a point linked to a referential of said first femur for which an optimization criterion taking into account said constraint is reached.

2. The method of claim 1, further comprising the steps of:
immobilizing the second femur,
displacing the first femur and locating several ones of said first femur's positions,
searching invariants of this displacement, taking into account the fact that the centers of rotations of the first and second femurs are distant by a substantially constant length.

3. The method of claim 2, further comprising the step of locating, for each of the several positions of the first femur, the position of the second femur to accordingly correct the position of the center of rotation between the first femur and the iliac bone.

4. The method of claim 1, further comprising the steps of:
displacing the thigh so that said center of rotation moves along a trajectory which is clearly mathematically distinct from all other points of the lower femur portion; and
searching the center of rotation having a specific trajectory by an optimization method.

5. The method of claim 4, wherein the thigh is moved so that the knee follows a loop trajectory, whereby only the trajectory of the center of rotation will optimize a distance in the expression of which the number of loops and some of their mathematical characteristics will be involved.

6. The method of claim 4, further comprising:
decomposing the thigh motion in several elementary motions;
calculating, for each elementary motion, an optimal center of rotation and an optimized distance value;
statistically defining the center of rotation, taking into account each of the calculated center of rotation and the optimized distance value, obtained based on each of the elementary motions.

7. The method of claim 1, further comprising the steps of:
moving the thigh so that the thigh's lower, portion describes as simple a trajectory as possible, so that the searched center of rotation describes a mathematically simple trajectory; and
searching the center of rotation with a mathematically simple trajectory by an optimization method.

8. The method of claim 7, further comprising:
decomposing the thigh motion in several elementary motions;
calculating, for each elementary motion, an optimal center of rotation and the value of an optimized distance;
statistically defining the center of rotation by taking into account each of the calculated center of rotation and the value of the optimized distance, obtained based on each of the elementary motions.

9. The method of claim 1, further comprising the steps of:
performing a succession of elementary motions of the thigh;
searching the position of the center of rotation of the femur for each of the elementary motions assuming that said femur has remained fixed;
determining a confidence ellipsoid within which the probability of presence of the femur center of rotation is high; and
calculating the position of maximum probability of the femur center of rotation based on the confidence ellipsoids.

10. The method of claim 9, wherein some of the elementary motions of the thigh are performed in a plane and are of small amplitude.

11. The method of claim 9, wherein some of the elementary motions of the thigh are performed by rotating the femur around the femur's own axis.

12. A device for determining the center of rotation of a femur with respect to the iliac bone, comprising:
means for locating several positions of the femur during motions thereof,
means for imposing a constraint to the motion of said center of rotation without immobilizing the femur, and
calculation means for searching a point linked to a referential of said femur for which a minimization criterion is reached, taking said constraint into account.
